(19) United States
(12) Patent Application Publication
(10) Pub. No.: US 2005/0106259 A1
(43) Pub. Date: May 19, 2005

(54) GRAS two acid low pH compound consisting of citric and hydrochloric or phosphoric acids

(76) Inventor: John Barclay Carter, Fort Walton Beach, FL (US)

Correspondence Address: John B. Carter, P.O. box 1311, Fort Walton Beach, FL, 32549, US

(21) Appl. No.: 10/967,751

(22) Filed: Oct. 18, 2004

Related U.S. Application Data
(60) Provisional application No. 60/517,797, filed on Nov. 5, 2003.

(51) Int. Cl. A61K 31/19; A61K 33/18; A61K 33/42

(52) U.S. Cl. 424/601; 424/666; 514/574

(57) ABSTRACT

This invention has a focus of adaptability for the purpose of environmental preservation while satisfying the need for a low pH compound. This invention is a more environmentally friendly low pH formula for decontamination. It is an antimicrobial agent low pH acidic aqueous solution consisting of two acids with a pH of to less than one. It is non-toxic to humans and animals. Currently available formulas consist of three or more acids; however, the resulting disposal of this invention is a less objectionable pollutant into the environment. In areas that are concerned about phosphoric residue, the formula containing hydrochloric acid would be used and visa-versa. This invention is intended for use as a disinfectant in a food production, agriculture, household, animal care, cosmetic, medical, industrial, military, public safety, transportation, hygiene, locations of population concentration and institutions of confinement. This invention is intended for use alone in full concentration, diluted or as a component of other solutions. This invention also relates to the dilution and application methods of this invention.

### CROSS-REFERENCE TO RELATED APPLICATIONS

The application claims priority to U.S. provisional patent application, entitled “GRAS Two Acid Low pH Compound consisting of Citric and Hydrochloric or Phosphoric Acids,” Ser. No. 60/517,797, filed Nov. 5, 2003 by the present inventor.

### FEDERALLY SPONSORED RESEARCH

Not Applicable

### SEQUENCE LISTING OR PROGRAM

Not Applicable

### BACKGROUND OF THE INVENTION-FIELD OF INVENTION

This invention relates to low pH compositions, specifically for use as an anti-microbial non-caustic to humans and animals and is relatively environmentally friendly.

### BACKGROUND OF THE INVENTION-PRIOR ART

Low pH compositions and solutions containing such compositions are widely used. They are used in industry, food processing, sterilization and cleaning. Currently available compositions are made of three or more acids. Some are dangerously caustic to humans and animals. Some are non-caustic to humans but contain components that are not generally recognized as safe (GRAS) by the U. S. Food and Drug Administration. Others that are made of GRAS components contain objectionable pollutants.

Phosphoric residue as a pollutant in stream and rivers contributes to eutrophication. The end of the life cycle of any product is disposal. Products with phosphoric residue used in small quantities will have minimal adverse effect on the environment. If that product is used in large quantities by many households and/or in great quantity by industry the environmentally damaging effects could be profound.

This invention has a focus of adaptability for the purpose of environmental preservation while satisfying the need for a low pH compound. This invention is a more environmentally friendly low pH formula for decontamination. It is an anti-microbial agent low pH acidic aqueous solution consisting of two acids with a pH of to less than one. It is non-toxic to humans and animals. In areas that are concerned about phosphoric residue, the formula containing hydrochloric acid would be used and visa-versa. The resulting disposal of this invention is a less objectionable pollutant into the environment. This invention is intended for use as a disinfectant in food production, agriculture, household, cosmetic, medical, industrial, military, public safety, transportation, waste treatment, hygiene, locations of population concentration and institutions of confinement.

This invention is intended for use alone in full concentration, diluted or as a component of other solutions. This invention also relates to the dilution and application methods of this invention.

### BACKGROUND OF INVENTION-OBJECTS AND ADVANTAGES

This invention has the flexibility of providing a low pH solution while accommodating environmental needs. This invention will satisfy the need for low pH solutions and protect bodies of water. The eutrophication of bodies of water results from the increase of nutrients such as phosphorus causing increases in photosynthetic algae and bacteria lowering the waters oxygen level thereby resulting in fish kills. It will provide a means of decontamination and sterilization for homes, offices, industrial facilities, restaurants, medical facilities, military installations, prisons, cruise ships and other places people congregate. It also provides a means of decontamination and sterilization for animal cages and facilities. It is nontoxic and ingestible therefore, no protective gear or special skills are required. It is ingestible therefore, it can be used in food and drink products. Its residue on food is not a deterrent, it in fact kills transmittable microbial. It will provide room temperature sterilization for health care instruments thereby reducing burn risk to employees from harsh chemicals and/or autoclave. Medically, either alone or as an additive, it will treat wounds, sexually transmitted diseases, burns, infections and digestive disorders. It will kill germs in fabrics without bleaching colors. It will kill odors from urine by killing germs causing the odor rather than masking the odor. It can satisfy industrial needs for low pH solutions without the associated risk to employees and customers. It provides a skin wash for personnel after contact with persons, animals or items that may be infectious. Because it is nontoxic, it can be shipped in high concentration and diluted to the required level at its destination thereby reducing shipping cost in labor, fuel and traffic congestion. Insurance costs should be reduced because an accidental spill would not be a catastrophic danger to the public.

Examples of use include but are not limited to the following:
Sterilization of surgical theaters
Sterilization of medical equipment
Sterilization of biological waste for sanitary disposal
Sterilization and deodorization of veterinary clinics
Countermeasure to chemical and biological warfare
Disinfect and speed healing for burn victims
Disinfect and speed healing athlete's foot
Sterilization of drinking water
Food preservation
Household cleaner that is not dangerous to children and pets
Glass cleaner
Removal of oxidation from a variety of metals
Deodorizer and sanitize urine stains and pet odor
Additive to cosmetics and personal deodorants
Sterilization of tattoo parlor equipment
Sterilization of manicure equipment
Deodorize clothing and shoes
Cleaning build up in industrial process and transportation equipment
As a disinfecting rinse for food during processing
Sanitizing restaurants both in public and food preparation areas
Flushing heating and cooling systems to thwart “Legionnaire's Disease”
Decontamination after a chemical spill
Disinfecting for first responders rendering aid to HIV patients
Disinfecting public areas during the cold and flu season would reduce absenteeism.
Control swimming pool pH without dangerous chemicals
Paper pulp processing
Breaking down sewage waste reducing eutrophication

### SUMMARY

In summary this invention is a low pH solution that is not harmful to people or animals. It has vast applications in environmental sterilization, food production, health care and industry. It is more environmentally friendly than any similar product.

### DRAWINGS

None

### SPECIFICATIONS

Mixing should be done in a ventilated area.

The compound containing hydrochloric acid is made in the following manner. Quantities are approximate and dependent upon desired pH level and the stringency of the consumer's demand for quality consistency. This formula will produce approximately one U. S. Gallon with a pH of approximately 0.7. Into 115 fluid oz of water dissolve 8 oz by weight granulated citric acid. Stir. Add 8.5 fluid oz hydrochloric acid. Stir. The pH of the component could be raised by dilution of the compound with water to the minimal pH level to satisfy the use need and reduce the cost of the application.

The compound containing phosphoric acid is made in the following manner. Quantities are approximate and dependent upon desired pH level and the stringency of the consumer's demand for quality consistency. This formula will produce approximately one U.S. Gallon of compound with a pH of approximately 2.0. Into 115 fluid oz of water dissolve 8 oz by weight granulated citric acid. Stir. Add 6.0 fluid oz phosphoric acid. Stir. Increasing the amount of phosphoric acid can lower the pH. The pH of the component could be raised by dilution of the compound with water to the minimal pH level to satisfy the use need and reduce the cost of the application.

Although the description above contains specificities, these should not be construed as limiting the scope of the invention but as merely providing illustrations of some of the presently preferred embodiments of this invention. The scope of the invention should be determined by the appended claims rather by the examples given.

1. An aqueous acidic solution for sterilization and microbial growth inhibition consisting of two acids that is ingestible and thereby safe for use in environment products, food and drink production/processing and pharmaceutical products, said acid core composition prepared by the steps of:
creating an organic acid solution by dissolving granulated citric acid in water
then admixing an inorganic acid either hydrochloric or phosphoric acid mix to a homogeneous consistence.

2. The acidic solution of claim 1 wherein the second acid is situational determined by environmentally disposal preference.

3. The acidic solution of claim 1 wherein the first, second and second alternative acids are recognized by the Food and Drug Administration (FDA) as “Generally Regarded As Safe” (GRAS).

4. The acidic solution of claim 1 wherein the solution is substantially non-corrosive to metals.

5. The acidic solution of claim 1 wherein the solution is innocuous to humans and animals.

6. The acidic solution of claim 1 wherein the solution is safe for use on and in ingestible products.

7. The acidic solution of claim 1 wherein the solution is safe for use on surfaces that contact ingestible products.

8. The acidic solution of claim 1 wherein the solution is safe for use in wound treatment.

9. The acidic solution of claim 1 wherein the solution is safe for use in decontamination.

10. The acidic solution of claim 1 wherein the solution is an effective pharmaceutical additive.

11. The acidic solution of claim 1 wherein the solution has a pH to less than one.

12. The acidic solution of claim 1 wherein the solution pH can be adjusted by dilution or ratio of mix to the desired pH level to satisfy the use need.

13. The acidic solution of claim 1 wherein the solution may be applied by methods to include but not limited to: as additive to other products, immersion, flush, spray and transfer application i.e. towel, brush, sponge.
